(19) United States
(12) Patent Application Publication
(10) Pub. No.: US 2010/0121381 A1
(43) Pub. Date: May 13, 2010

(54) SURGICAL METHOD AND APPARATUS FOR TREATING SPINAL STENOSIS AND STABILIZATION OF VERTEBRAE

(75) Inventors: Scott Collins Berta, Foster City, CA (US); Michael Anthony Vaninetti, Palo Alto, CA (US); Shivanand Lad, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Springback, Inc., Palo Alto, CA (US)

(21) Appl. No.: 12/480,534

(22) Filed: Jun. 8, 2009

Related U.S. Application Data
(60) Provisional application No. 61/131,427, filed on Jun. 9, 2008.
(60) Provisional application No. 61/132,978, filed on Jun. 23, 2008.
(60) Provisional application No. 61/135,161, filed on Jul. 17, 2008.
(60) Provisional application No. 61/201,657, filed on Dec. 15, 2008.

(51) Int. Cl. A61B 17/70; A61B 17/88

(52) U.S. Cl. 606/264; 606/246; 606/279

(57) ABSTRACT

Disclosed is a prosthetic device for distracting spinal column segments in the lumbar and the lumbar-sacral regions comprising a first engagement arm, a second engagement arm, a coupling mechanism and a locking mechanism. The first and the second engagement arms are configured to receive a lamina portion of the spinal column segment. The coupling mechanism is disposed between the first and the second engagement arms and is configured to allow the device to transition from an unextended configuration to an extended configuration in order to distract the spinal column segment. The locking mechanism is configured to maintain the extended configuration of the device.

### CROSS-REFERENCES TO RELATED APPLICATIONS

This application claims priority to provisional applications 61/131,427 filed on Jun. 9, 2008; 61/132,978 filed on Jun. 23, 2008; 61/135,161 filed on Jul. 17, 2008; and 61/201,657 filed on Dec. 15, 2008.

### BACKGROUND OF THE INVENTION

1. Field of the Invention

This invention relates generally to spinal prosthetic devices and more specifically to apparatus and approaches for interlaminar process, interspinous process, and spinolaminar junction distraction and stabilization for treatment of spinal stenosis.

2. Description of the Related Art

The spine and its components can become damaged through disease, injury, or natural degeneration. In such cases, the vertebrae no longer articulate or properly align with each other. This can result in deviation from the normal spinal structure, loss of mobility, and pain or discomfort. For example, degenerative phenomena such as spinal stenosis, spondylosis, spondylolisthesis, or osteoarthritis may cause back pain, such as lower back pain localized in the lumbosacral region. Such phenomena may be caused by a narrowing of the spinal canal by pre-existing congenital conditions or injuries such as ligamentum flavum hypertrophy, intervertebral disc bulging or herniation, and facet thickening with arthropathy of the capsule soft tissues that result in the pinching of the spinal cord and/or nerves in the spine. Indeed, lumbar spinal stenosis is a common reason for surgery of the spine in patients over the age of 65. The relevance in the geriatric population makes traditional surgical treatment of spinal stenosis particularly difficult because these patients are at a significantly increased surgical risk because of their pre-existing medical conditions or history.

The traditional treatment of spinal stenosis consists of an extensive resection of posterior spinal elements. Additionally, wide muscular dissection and retraction is usually employed to achieve adequate visualization during surgery. Various operative techniques have been used for decades with varying degrees of success. The surgical process and the attendant manipulation of the spine and the tissue surrounding it can also be associated with significant operative blood loss as well as prolonged post-operative pain and weakness at the surgery site. Further, iatrogenic injuries can lead to paraspinal muscle denervation and atrophy, which may correlate with an increased incidence of “failed back syndrome” and chronic pain. Because patients who have stenosis are usually elderly and medically frail, these injuries often cause one or more post-operation complications and a prolonged recovery time.

The current management of such spinal conditions may also include the use of prosthetic devices. In all such devices, it is essential to securely anchor the device to the vertebra while not damaging it. It is also desirable to minimize the requisite surgery to place the device in the patient. Furthermore, it is desirable for the device to contain minimal working or moving parts since a complex system could be prone to malfunction and may require more invasive surgery for insertion and calibration. Additionally, it is desirable for a prosthetic device to be able to distract both adjacent and nonadjacent vertebrae and to be able to be functional between the lumbar and the sacral portions of the spine.

Present spinal prosthetic devices do not address many of the desirable characteristics mentioned above. For example, many of the current prosthetic devices contain multiple moveable parts that could be prone to mechanical malfunction and may require complex insertion procedures and calibrations. U.S. Pat. No. 4,611,582 to Duff, for example, discloses a device consisting of moveable vertebral clamps that hinge on a ball-and-socket mechanism disposed on a moveable body. The device has to be correctly calibrated in order to control the spatial relationship between the clamps, and thereby between the vertebrae. The Duff device also contains multiple moveable sub-parts that have to be individually calibrated and secured by screws. This system is both complex and could be prone to malfunction. Similarly, U.S. Pat. No. 7,491,238 to Amin et al., discloses a large and complex apparatus comprising multiple moveable mechanical arms secured to multiple spinal structures by fasteners such as screws. The Arnin device is large and contains multiple adjustable parts which could result in complicated insertion surgery requiring extensive manipulation by the surgeon. Similarly, U.S. Pat. No. 7,011,658 to Young discloses a device with opposite first and second engagement ends and a screw-based mechanism for moving the opposite engagement ends in extension and refraction. The Young device additionally utilizes a complex locking and driving mechanism that contains multiple screws and pins that could also complicate the insertion surgery. Similarly, U.S. Pat. No. 5,007,909 to Rogozinski discloses multiple clamps and a rod that are affixed to the lamina of the vertebra, where each clamp is affixed to the vertebrae and the rod through a complex assembly. Given the fact that the Rogozinski device requires multiple clamps and that each clamp has to be calibrated to the vertebrae and the rod, it is likely that a complex insertion and calibration procedure is required. Also, U.S. Pat. No. 4,697,582 to Williams discloses a mechanical assembly with retaining clamps, where each clamp is screwed onto the vertebra and an elastic structure is attached to the retaining clamp fixed to each vertebra. Similar to the Rogozinski device, the Williams device also utilizes multiple clamps where each clamp has to be individually screwed into the vertebrae.

Furthermore, many of the current devices are large or their insertion surgical techniques could result in higher risk of post-operative complications. For example, U.S. Pat. No. 7,052,497 to Sherman et al. discloses a loading device that requires two different surgeries for proper insertion and calibration. The multiple surgeries required by the Sherman device could result in increase in recovery time and post-operation complications and also increase the medical cost associated with each surgery. Similarly, U.S. Pat. No. 5,540,688 to Navas discloses a device in the form of a damper attached to two anchor implants that are screwed into two adjacent vertebrae. The need for screwing both ends of the Navas device into the vertebrae complicates the surgical procedure and is likely to cause greater damage to the surrounding tissues.

Also, many of the current devices are impractical to function between non-adjacent vertebrae. For example, U.S. Pat. No. 5,415,661 to Holmes discloses a flexible implantable device that is fitted in between, and screwed on to adjacent vertebrae. Given that the device needs to be fitted between two adjacent vertebrae, it is unlikely to function successfully on non-adjacent vertebrae.

In addition to the abovementioned limitations, it is impractical for many of the current spinal prosthetic devices to perform distraction on the lumbosacral level. For example, U.S. Pat. No. 5,645,599 to Samani discloses a device comprising a U-shaped body and two pairs of brackets that are fixed to spinous process of adjacent vertebrae. U.S. Pat. No. 6,074,390 to Zucherman et al. discloses a spine distraction implant that alleviates pain associated with spinal stenosis by inserting the device between affected adjacent vertebrae by means of telescoping fork ends, where the fork ends brace the spinous process. Both the Zucherman device and the Samani device are specifically structured to receive the spinous process. However, it is less desirable for either of the devices to be used at the sacral level since the sacrum lacks a significant spinous process to allow for proper docking and distraction.

Given the limitations of traditional surgical treatments and current prosthetic devices, there is a need for a novel prosthetic device that embodies the above desirable qualities. Such a device should contain minimal working parts. It should also be insertable with minimally invasive procedure and should be easy to manufacture. It should achieve decompression and alleviation of pain on the lumbosacral junction and be structured to function on adjacent and non-adjacent vertebrae. At least some of these objectives will be addressed by the present invention.

### BRIEF SUMMARY OF THE INVENTION

The present invention is a prosthetic device for distracting and stabilizing spinal column segments in the lumbar and the lumbar-sacral regions by engaging the spinal column segments. Engagement occurs with two engagement arms that are configured to distract the targeted spinal segments to relieve pain and discomfort associated with spinal stenosis or other spinal disorders.

In one embodiment, the prosthetic device comprises a first engagement arm and a second engagement arm wherein each of the engagement arms terminates in a lamina receiving configuration that comprises at least two tines that are configured to receive a lamina portion of the spinal column segment. The engagement arms may further comprise a secondary branch or an opening to receive an attachment screw configured to attach to a vertebra. Additionally, the preferred embodiment may be further secured to a lamina portion by a safety band or a screw.

The device further comprises a coupling mechanism disposed between the first and the second engagement arms. The coupling mechanism is configured to allow the device to transition from an unextended configuration to an extended configuration in order to distract the spinal column segment. The coupling mechanism of the preferred embodiment may be a joint such as a pivot joint, a revolute joint, a pin joint, or a hinge joint. The coupling mechanism may also be a telescoping mechanism, a spring, a rotation mechanism or a slidable mechanism.

Also, the device comprises a locking mechanism that is configured to maintain the extended configuration of the device. The locking mechanism of the preferred embodiment may be a self-locking joint, a fastening screw and an opening configured to receive the fastening screw or a sleeve configured to be placed over the coupling mechanism.

Additionally, multiple devices may be used. For example, the devices may be used as part of aa bilateral system comprising two devices working together to distract two or more spinal column segments. Such a bilateral system may further comprise a connecting mechanism such as a U-hook, a process pin or a safety band.

Other aspects of the invention include methods corresponding to the devices and systems described above. Such methods include the steps of positioning and securing the prosthetic device by fitting lamina portions of the first and the second spinal portions between the tines of the engagement arms. The preferred embodiment further includes the step of operating the coupling mechanism to extend the first and second engagement arms such that the engagement arms engage with the lamina portions of the first and the second spinal portions to distract the spinal column segment. Furthermore, the preferred embodiment includes the step of operating the locking mechanism by engaging a self-locking joint, inserting a fastener screw into a receiving hole or placing a sleeve over the coupling mechanism in order to maintain the distraction. Additionally and optionally, the method includes the pre-insertion step of separating the ligamentum flavum from the spinal column segments, preparing the lamina portions of the spinal column segments to receive the device and sizing the spinal column segments to determine the desired degree of distraction. Additionally and optionally, the method further includes the step of using the device as a fusion adjunct to supplement posterolateral fusion.

### BRIEF DESCRIPTION OF THE DRAWINGS

FIG. 1 shows a posterior view of the device engaging at L4 superiorly and S inferiorly on the right and at L5 superiorly and S inferiorly on the left.

FIG. 2A shows a lateral view of the device.

FIG. 2B shows a lateral view of the device comprising a joint mechanism in partially collapsed and expanded configurations.

FIG. 2C shows the device with at least one dynamic portion.

FIG. 2D shows an embodiment of the device comprising a slidable mechanism.

FIG. 2E shows an embodiment of the device comprising a telescoping mechanism.

FIG. 2F shows an embodiment of the device comprising a spring.

FIG. 3A shows a locking mechanism which uses a screw-type fastener to lock the joint.

FIG. 3B shows a sleeve which can slide onto the hinge portion of the device to lock the hinge.

FIG. 4A shows the inferior and superior engagement arms of the device from a lateral view engaging on L4 and S.

FIG. 4B shows the device in operation. The device starts out in a contracted configuration, wherein the inferior and superior engagement arms of the device engage and distract L5 and S1 spinal column segments.

FIG. 4C shows two devices operating together to distract two spinal column segments.

FIG. 5 shows safety bands used to secure the device to the spinal column segment to inhibit movement of the device once implanted.

FIG. 6A shows different configurations of the engagement arms.

FIG. 6B shows one embodiment of the device comprising engagement arms with multiple tines at various orientations.

FIG. 6C shows an alternative embodiment of the device comprising one engagement arm with four tines and a second engagement arm with a fan shaped tine.

FIG. 6D shows the lateral view of an alternative embodiment of the device where one engagement arm with four tines engages L5 and the other engagement arm with two tines engages S1.

FIG. 7 shows different possible curvatures of the device.

FIG. 8 depicts an alternative embodiment for locking the device in place once implanted.

FIG. 9 depicts an alternative embodiment for locking the device in place once implanted.

FIG. 10 shows an alternative embodiment that comprises a screw attachment and a multi-branched tine arrangement.

FIG. 11 shows one embodiment in which one engagement arm comprises a pin assembly while the other engagement arm comprises two moveable tines connected by a pivot point.

FIG. 12 shows one embodiment in which one engagement arm comprises a pin assembly while the other engagement arm comprises two fixed tines.

FIG. 13 shows one embodiment in which both engagement arms comprise pin assemblies.

FIG. 14A shows one embodiment in which both engagement arms comprise clamp-like configurations.

FIG. 14B shows one embodiment in which one engagement arm comprises a clamp-like configuration while a second engagement arm comprises a tine configuration.

FIG. 15 shows one embodiment in which the device is single bodied with no moveable parts.

FIG. 16A shows an embodiment of the device comprising flared ends.

FIG. 16B shows an embodiment of the device comprising non-flared ends.

### DETAILED DESCRIPTION OF THE INVENTION

Although the detailed description contains many specifics, these should not be construed as limiting the scope of the invention but merely as illustrating different examples and aspects of the invention. It should be appreciated that the scope of the invention includes other embodiments not discussed in detail above. Various other modifications, changes and variations which will be apparent to those skilled in the art may be made in the arrangement, operation and details of the methods and systems of the present invention disclosed herein without departing from the spirit and scope of the invention as described.

The present invention is a prosthetic device for distracting spinal column segments in the lumbar and the lumbar-sacral regions by engaging the spinal column segments. Engagement occurs with two engagement arms that are configured to distract the targeted spinal segments to relieve pain and discomfort associated with spinal stenosis or other spinal disorders. Although the device is used for distraction and stabilization, it is contemplated that the device may also be used for inhibition of spinal flexion through attachment around the spinous process and the laminar process.

The device may be inserted through a minimally invasive incision and extended after engaging a portion, for example the lamina, of the spinal column segment. The act of extending the device will serve as an internal distraction mechanism for alleviating spinal stenosis by opening the neural foramina in between the segments of interest. This device can also be customized to a patient's degree of stenosis using a variable adjusting portion.

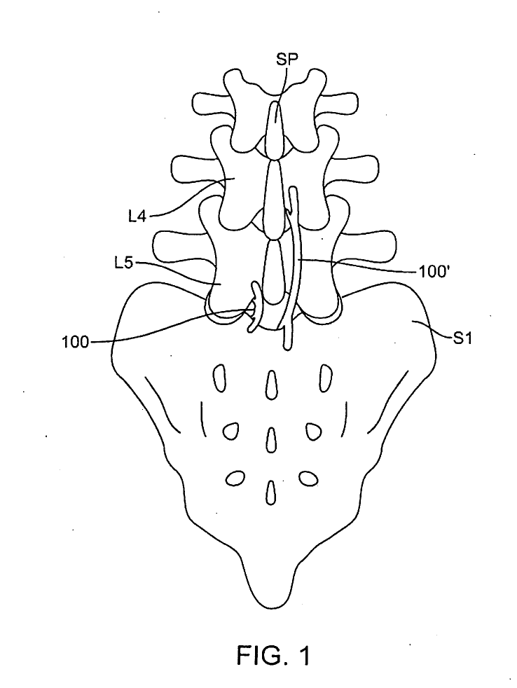

Referring to the figures, FIG. 1 shows one embodiment of the device oriented within the spinal column. The device 100 can be inserted between adjacent spinal column segments such as the lumbar vertebra five L5, and the sacral vertebra one S1. Alternatively the device 100′ can be inserted between non-adjacent spinal column segments such as the lumbar vertebra four L4 and the sacral vertebra one S1. In this embodiment the device engages with at least one lamina portion of the spinal column segment without disturbing the spinous process SP. For purposes of this description, the term lamina portion is used interchangeably with the terms lamina, and laminar process. Further, for the purposes of this description, all spinal vertebrae, including the sacrum, include a lamina portion that is. Although the device is used for lumbar spinal column segments as well as the lumbosacral junction, it is anticipated that the device may also be used in any spinal column segments, including the cervical and thoracic spinal column segments as well as at the occipital cervical junction.

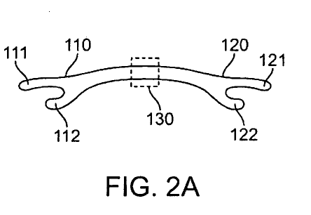

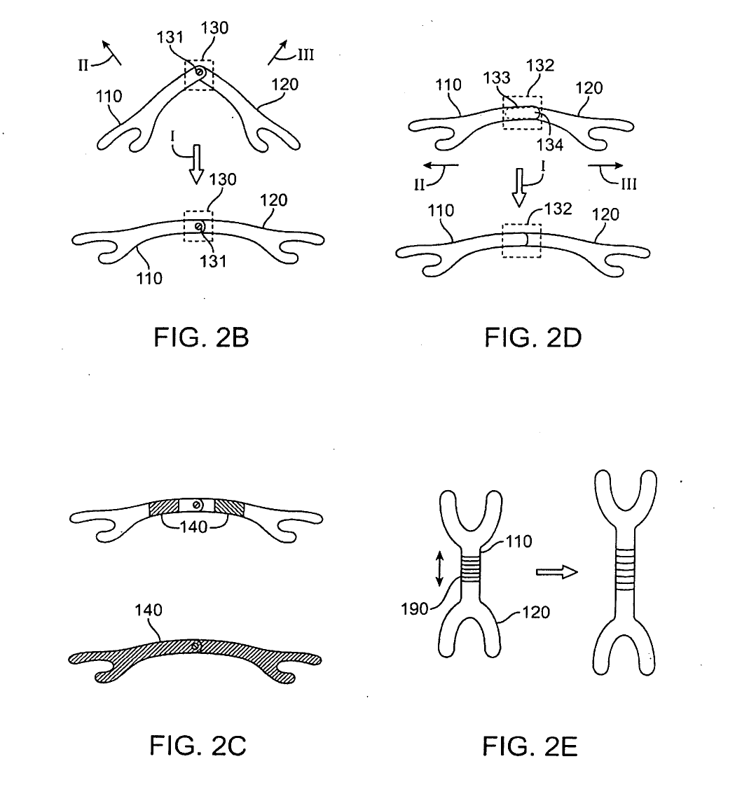

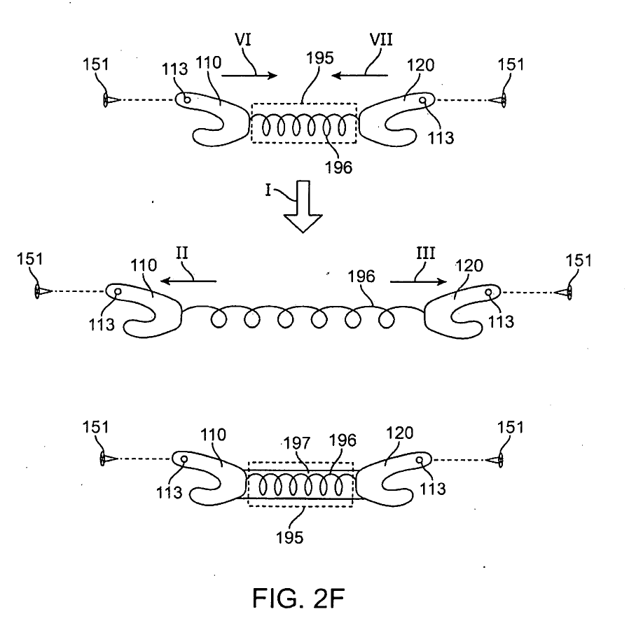

FIG. 2A shows one embodiment of the device comprising a first engagement arm 110, a second engagement arm 120, and a coupling mechanism 130 disposed between the first engagement arm 110 and the second engagement arm 120. The first engagement arm 110 terminates in a lamina receiving configuration that comprises at least one projection such as a tine. For example as shown in FIG. 2A, the device comprises a first tine 111 and a second tine 112 (which together form a clevis with an intermediate region shaped to receive the lamina). The tines may be of various shapes, sizes, or lengths. As shown in FIG. 2A, for example, the first tine 111 is longer than the second tine 112. Similar to the first engagement arm, the second engagement arm 120 terminates in a lamina receiving configuration that comprises at least one projection such as a tine. As shown, the second engagement arm comprises tines 121 and 122. These tines too may be of various shapes, sizes, and lengths. As shown in FIG. 2A, for example, the first tine 121 may be longer than the second tine 122. Furthermore, the size, shape, or length of the tines 111 and 112 are independent of the size, shape, or length of tines 121 and 122. In certain specific embodiments, however, the devices of the present invention will generally be symmetrical on each side being formed as mirror images with the structure joining the tines being straight or slightly arched or alternatively, being resilients joined as illustrated in later embodiments.

FIG. 2B shows one embodiment of the device where the first engagement arm 110 and the second engagement arm 120 are connected by a coupling mechanism 130. In this embodiment, the coupling mechanism 130 comprises a joint 131. The joint allows the first engagement arm 110 and the second engagement arm 120 to transition to an extended configuration as indicated by arrow I by unfolding away from each other in the direction indicated by the arrows II and III while maintaining the structural integrity of the device. In FIG. 2B, a hinge joint is shown. However, the joint may be a pivot joint, a revolute joint, a pin joint, or any other joint configured to foldably connect the engagement arms 110 and 120.

As shown in FIG. 2C, the device optionally further comprises at least one dynamic portion 140 that is capable of contracting or expanding upon the application of biomechanical force within the body, thereby providing flexibility while maintaining distraction of spinal column segments. The dynamic portion may damp, absorb, or otherwise reduce the impulse created by the biomechanical force The dynamic portion may comprise a defined area of either one or both of the two engagement arms, or the dynamic portion may comprise the entirety of either one or both of the two engagement arms. The dynamic portion may also be incorporated onto a surface of either one or both of the two engagement arms. The dynamic portion may comprise any or a combination of a variety of biocompatible dynamic materials, such as polyetheretherketone (PEEK). In addition, a variety of growth factors, such as bone morphogenetic protein (BMP), may be incorporated into the device as a fusion adjunct to stimulate bone deposition and growth. Optionally, the device may also be made of a bioresorbable material such as polylactic acid (PLA). Furthermore, the entirety or a portion of one or more engagement arms may be made of or be coated by a material to provide better docking or interfacing between the device and the vertebrae. This material may be compliant, and formed from such materials as rubber or plastic.

FIG. 2D shows another embodiment of the device where the first engagement arm 110 and the second engagement arm 120 are connected by a coupling mechanism comprising a slidable mechanism 132. The slidable mechanism 132 comprises a first sliding end 133 and a second sliding end 134 configured to slide relative to each other to extend the device. As shown in FIG. 2D, the first end 133 is a male end and the second end 134 is a female end joined in a telescoping manner. Alternatively the first and second sliding ends may comprise a side-by-side sliding mechanism, for example using tracks or brackets to join the arms together. As shown in FIG. 2D the device starts out in an axially contracted configuration. During or after insertion between the spinal column segments, the engagement arms are pulled axially apart in the direction of the arrows II and III so that the two engagement arms 110 and 120 slide and extend away from each other, thereby transitioning the device to an extended configuration as indicated by arrow I. At the extended configuration, the slidable mechanism 132 optionally snaps into position using a detent or other locking mechanism to maintain the extended configuration. Optionally, in this embodiment, the device may include springs or other resilient mechanism to provide dynamic characteristics wherein the sliding ends may be able to slide within a range after insertion without bending the device.

FIG. 2E shows another embodiment of the device comprising a first engagement arm 110, a second engagement arm 120, and a coupling mechanism comprising a telescoping mechanism 190. The telescoping mechanism 190 may comprise a bolt and screw expanding assembly wherein by turning the bolt, the assembly expands the body of the device. Alternatively, the telescoping mechanism may be any assembly that is able to elongate the body of the device such as a rotation mechanism that is effected by turning or rotating the engagement arm(s).

FIG. 2F shows another alternative embodiment of the device comprising a first engagement arm 110, a second engagement arm 120, and a coupling mechanism comprising a spring 195. The spring 195 comprises a spring 196. The device is first compressed into the compressed state as shown by arrows VI and VII to load the potential energy in the spring. Compression may be achieved by using one of various compression tools known by those skilled in the art. Alternatively, the spring is pre-compressed during manufacturing. During insertion, the spring is released to transform the device into the extended state as shown by arrow I by pushing the two engagement arms 110 and 120 away from each other in the directions shown by arrows II and III. Additionally and optionally, the spring 195 may comprise a spring 196 housed within a sleeve 197. The sleeve 197 may be a sliding tube, sheath or other configuration made of compressible material.

Additionally and optionally, to further enhance device stability and retention, the first and the second engagement arms may be secured to the spinal column segments by using screws 151 that are received by holes 113, which are located on the engagement arms.

Although the coupling mechanisms described above are described with respect to one embodiment of the device, it should be noted that any coupling mechanism may be used in conjunction with any embodiment described herein.

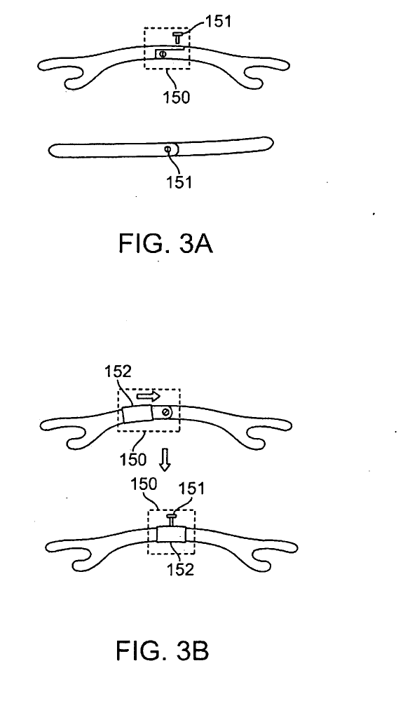

Additionally, as shown in FIG. 3A the device comprises a locking mechanism 150. In one example embodiment, the locking mechanism 150 is a self-locking joint comprising a self-locking assembly. In such an embodiment, when the foldable arms engage with and distract the spinal column segments, the self-locking joint assembly locks automatically upon fully unfolding the arms. Additionally and optionally the locking mechanism may comprise a fastener screw 151 received by a hole in the device (hole not shown). Furthermore, the locking mechanism may comprise a fastener screw independent of the self-locking joint. The fastener screw may be of any shape, size, or design as long as the screw is configured to be inserted into the device without disturbing the interspinous ligament. The fastener screw may be made of titanium or any other biocompatible material.

Alternatively, as shown in FIG. 3B, the device comprises a locking mechanism 150 and a moveable sleeve 152. The moveable sleeve can be placed or slid over the point of extension such as the joint or the contact position of the sleeve. Additionally and optionally, the sleeve may be further secured by a fastener screw 151. The sleeve may be of any shape, size, length, or design as long as the sleeve covers the coupling mechanism in a manner that inhibits the coupling mechanism from moving. The sleeve may be made of titanium or any other biocompatible material.

Although the locking mechanisms described above are described with respect to one embodiment of the device, it should be noted that any locking mechanism may be used in conjunction with any embodiment described herein. For example, locking mechanisms such as one or more turn-key mechanisms, a clamp or a clasp, may be used to maintain the expanded configuration of the device.

Prior to insertion of the device, a separating device (not shown) may be used to separate the ligamentum flavum from the lamina portion of the spinal column segment. Furthermore, a sizing device (not shown) may be used to determine the desired degree of distraction. Optionally, the lamina portion of the spinal column segment may be prepared to receive the device by creating an indentation at the engagement site on the lamina portion by using a rongeur. Also, the desired degree of distraction may be first achieved by using tools and methods known in the art before the device is inserted.

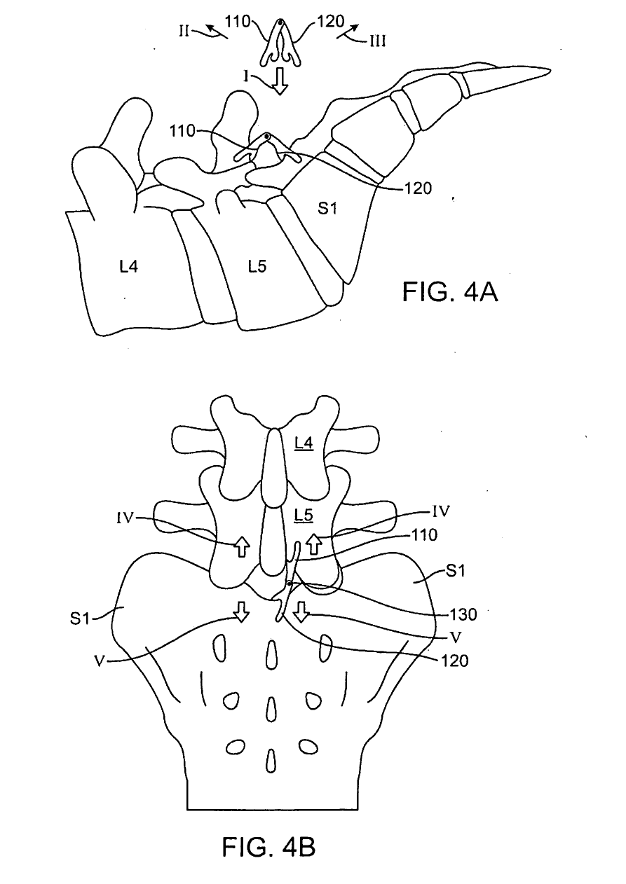

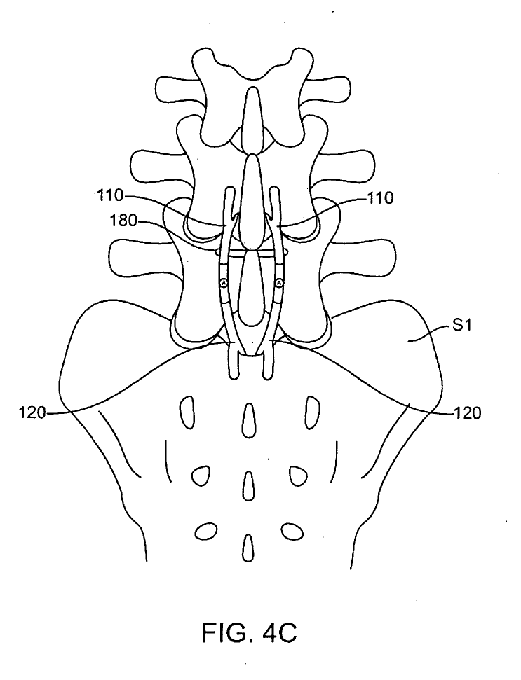

FIG. 4A shows the device being inserted into the spinal column as exemplified by arrow I. During insertion, as shown by arrow II and arrow III, the first engagement arm 110 and the second engagement arm 120 transitions from the unextended position to the extended position. For example, in the embodiment shown, the coupling mechanism comprises a joint wherein the unextended position is the folded position and the extended position is the unfolded position. Alternatively, if the coupling mechanism comprises a slide, the unextended position may be the shortened position and the extended position may be the expanded position. The device may be inserted manually or may be inserted by using a holder implement (not shown). The holder implement may be used to hold the device in the unextended configuration and positions the device for insertion. Once the device is inserted, the holder implement may be used to engage the device with the spinal column segments and transform the device to the extended position. The holder implement may comprise a handle of any appropriate size or shape. Optionally, the holder may further comprise magnetic prongs to hold the device during insertion.

FIG. 4B shows the device distracting the targeted spinal column segment where the first engagement arm 110 engages with the lamina portion of a spinal column segment such as L5. The second engagement arm 120 engages with a lamina portion of a different spinal column such as S1. The lamina portions engaged by the first and the second engagement arms 110 and 120 can be lamina portions of any sacral vertebrae, lumbar vertebrae, thoracic vertebrae, or cervical vertebrae. The coupling mechanism 130 disposed in between the first and second engagement arms is adjusted, for example, by pushing on the hinge joint to unfold the engagement arms 110 and 120. This alters the spatial relationship between the two spinal column segments by expanding the distance between the two spinal column segments. Alternatively, if a slidable or telescoping mechanism is used, the two engagement arms 110 or 120 are extended apart. Thus, the device is able to achieve distraction of the spinal column segments in the direction shown by arrows IV and V. The locking mechanism, if a self-locking mechanism is used, will automatically lock when the arms achieve their intended unfolded configuration. Additionally or alternatively, if a non-self locking mechanism such as a screw sleeve, or any other suitable locking mechanism, is used, it is then engaged to maintain the extended position of the engagement arms. Additionally, the device may achieve distraction to multiple levels of spinal column segments. For example, the engagement arms of the device may engage with L4 and S1 spinal column segments which results in distraction to both L4-L5 and L5-S1 junctions.

FIG. 4C shows two devices working together as a bilateral distraction system to provide additional distraction or support. The first engagement arms 110 of both devices are attached to one spinal column segment while the second engagement arms 120 of both devices are attached to the second spinal column segment. Two devices may be connected by a connecting mechanism 180 to provide additional support and stabilization. The connecting mechanism may be a U-hook, an interspinous process pin, a safety band or any other suitable connecting mechanism.

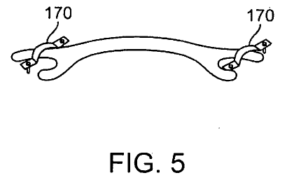

Additionally and optionally, to further enhance device stability and retention other approaches could be used for securing the device to the vertebrae. For example, the device may be secured by a safety band 170 as shown in FIG. 5. The safety band 170 is placed across a tine on the dorsal portion of the lamina, and the ends of the safety band 170 are secured to the vertebra. The band 170 may be of any shape, size, length, or design suitable to aid in securing the engagement arms to the spinal column segments. The safety band may be made of any biocompatible material. Additionally and alternatively, the engagement arms of the device can be configured to receive a screw, and the device may be screwed onto some portion of a vertebra.

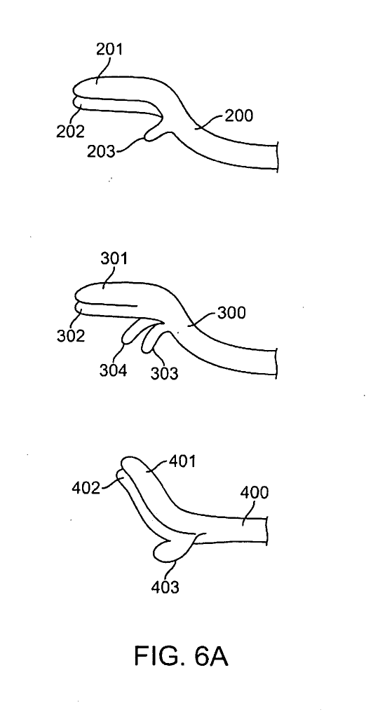

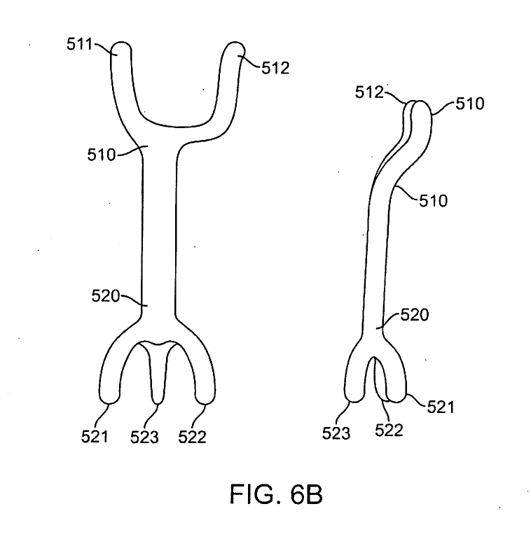

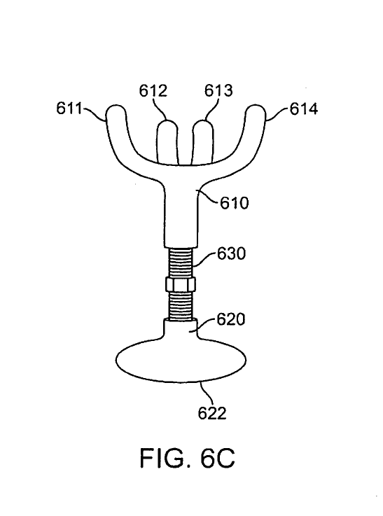

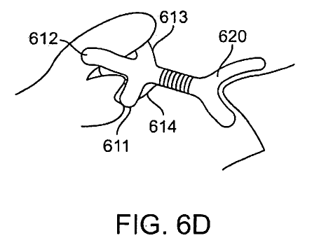

The number, size, shape, length, or orientation of the projections configured to receive a lamina may be specifically adapted or customized to best fit the targeted spinal column segment. For example, FIG. 6A shows three different configurations. The configurations shown comprises tines, however any projections may be used. In one configuration 200, an engagement arm terminates in a lamina receiving configuration that comprises three tines, a first tine 201, a second tine 202 and a third tine 203. The tines are configured such that the first and the second tines 201 and 202 are oriented dorsal to the spinal column segment (e.g., the lamina) when engaged while the third tine 203 is oriented ventral to the spinal column segment. In another configuration 300, an engagement arm terminates in a lamina receiving configuration that comprises four tines, a first tine 301, a second tine 302, a third tine 303 and a forth tine 304. The tines are oriented such that the first tine 301 and the second tine 302 are oriented dorsal to the spinal column segment (e.g., the lamina), whereas the third tine 303 and the forth tine 304 are oriented ventral to the spinal column segment. In another configuration 400, an engagement arm terminates in a lamina receiving configuration that comprises three tines, a first tine 401, a second tine 402 and a third tine 403. In this embodiment, the third tine 403 is configured to be broader than tines 401 and 402. The tines are configured such that the first tine 401 and the second tine 402 are oriented dorsal to the spinal column segment (e.g., the lamina), and the third tine 403 is oriented ventral to the spinal column segment.

One embodiment of the device comprises additional projections such as tines at various orientations as shown in FIG. 6B. The device comprises a first engagement arm 510 and a second engagement arm 520. The first engagement arm comprises a first tine 511 and a second tine 512, wherein the first and second tines 511 and 512 are configured to engage with a spinal column segment such as the spinous process. The second engagement arm comprises a first tine 521, a second tine 522, and a third tine 523. The tines are configured such that the first and the second tines 521 and 522 engage the dorsal portion of the spinal column segment (e.g., the lamina) while the third tine 523 engages the ventral portion of the spinal column segment.

Alternatively, as shown in FIG. 6C, the device comprises a first engagement arm 610, a second engagement arm 620, and a coupling mechanism 630. The first engagement arm 610 comprises projections such as a first tine 611, a second tine 612, a third tine 613 and a forth tine 614. The second engagement arm 620 comprises a first tine, not shown, and a second tine 622. The tines may vary in shapes, sizes, lengths or design. For example, the second tine 622 is shown comprising a broader configuration than the first tine (not shown). As shown in FIG. 6D, the first tine 611 and the forth tine 614 are oriented to ventrally engage a spinal column segment (e.g., the spinous process) wherein the second and the third tines 612 and 613 are oriented to dorsally engage the spinal column segment (e.g., the spinous process). The tines of the second engagement arm 620 are configured such that one of the tines is configured to fit dorsal to the spinal column segment (e.g., the sacral lamina) whereas the other tine is configured to fit ventral to said spinal column segment.

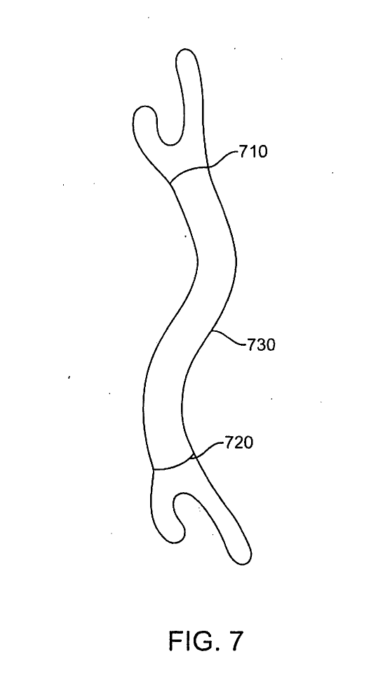

FIG. 7 shows another embodiment of the device comprising a first engagement arm 710, a second engagement arm 720, and a curved body 730 therebetween. The curved body may comprise double curves as shown, but may also comprise a single curve or multiple curves. The curved body can have various degrees of curvature to best fit the contour of the patient's body or to provide dynamic load-bearing support. It is further contemplated that any of the embodiments described herein may comprise a curved body.

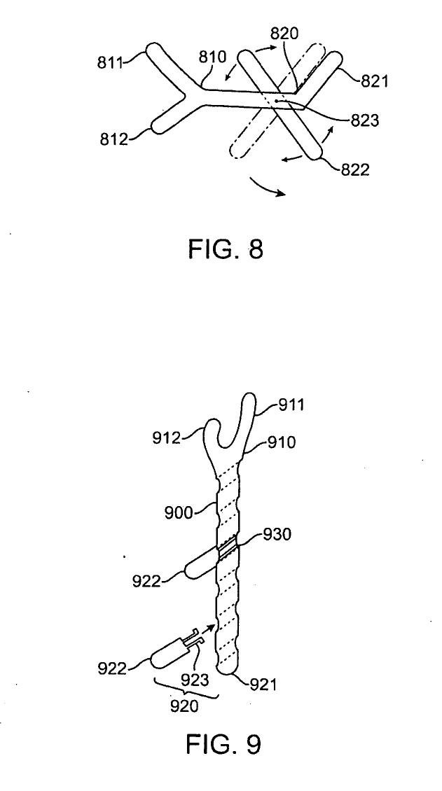

FIG. 8 shows yet another embodiment of the device comprising a first engagement arm 810 and a second engagement arm 820. The first engagement arm further comprises projections such as a first tine 811 and a second tine 812. Both the first tine 811 and the second tine 812 are configured to receive the lamina portion of a spinal column segment. The second engagement arm comprises a first tine 821 and a second tine 822. The first tine 821 has a fixed position, and the second tine 822 is configured to revolve around a self-locking pivot point 823. During insertion of the device into a patient's body, the first engagement arm 810 engages the lamina portion of a spinal column segment. The first tine 821 of the second engagement arm initiates the engagement of the lamina portion of a second spinal column segment by coming into contact with the lamina. Then, the second tine 822 of the second engagement arm revolves around the self-locking pivot point from a neutral position to an engagement position by coming into contact with the lamina. The self-locking pivot point then locks the second tine 822 in the engagement position and thus completes the engagement of the lamina portion of the second spinal column segment.

FIG. 9 shows another embodiment of the device comprising a first engagement arm 910 and a second engagement arm 920. The first engagement arm comprises projections such as a first tine 911 and a second tine 912. The tines 911 and 912 are configured to receive a lamina portion of a spinal column segment. The second engagement arm comprises a first tine 921 and a second tine 922. The first tine 921 has a fixed position, and the second tine 922 is a separate body insertable into the main body of the device 900, for example by using a pin 923 that is received by one of one or more outlets 930. During insertion, the first engagement arm 910 engages the lamina portion of a spinal column segment. The first tine 921 of the second engagement arm also engages a lamina portion of a second spinal column segment by contacting the lamina. Then, the second tine 922 of the second engagement arm 923 is inserted into the appropriate outlet 930, thus contacting a lamina portion. The pin 923 and outlet 930 then lock the second tine 922 and thus engage the lamina portion of the second spinal column segment.

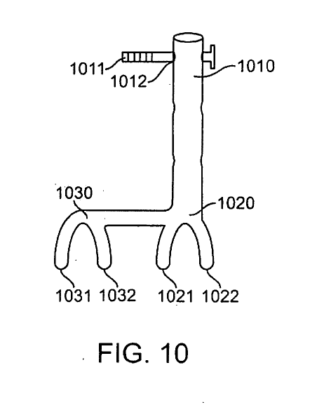

FIG. 10 shows another embodiment of the device comprising a first engagement arm 1010 and a second engagement arm 1020. The first engagement arm further comprises a fixation pin 1011 and a pin receiving opening 1012. The second engagement arm comprises a first tine 1021, a second tine 1022, and at least one additional branch 1030 comprising at least two tines, for example, a third tine 1031 and a forth tine 1032. During insertion of the device into a patient's body, the first engagement arm 1010 is fixed onto a spinal column segment by inserting the pin 1011 through the opening 1012 and securing the pin 1011 onto the spinal column segment. The second engagement arm 1020 engages the sacrum by receiving the lamina portion of the sacrum between tines 1021, 1022, 1031 and 1032.

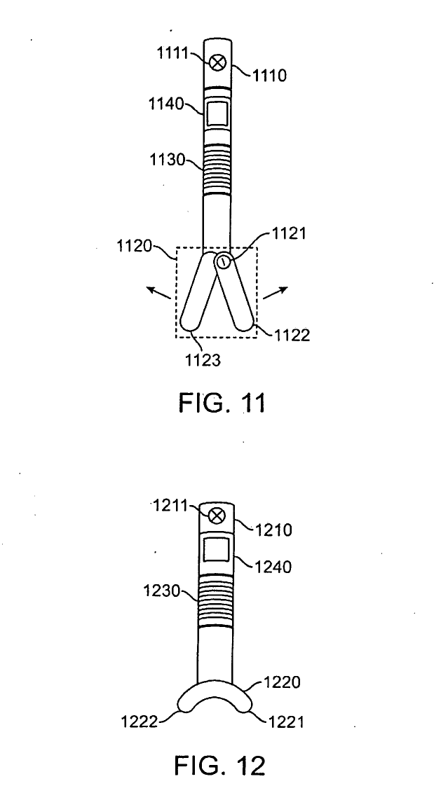

In an alternative embodiment, as shown in FIG. 11, the device comprises a first engagement arm 1110 and a second engagement arm 1120, a coupling mechanism 1130, and a dynamic portion 1140. The first engagement arm comprises a fixation pin 1111 and a receiving opening (not shown) that is configured to receive the fixation pin 1111. The second engagement arm comprises projections such as a moveable first tine 1122 and a moveable second tine 1123 connected by a pivot 1121 enabling the tines to move from an unfolded position to a folded position. During insertion of the device into a patient's body, the first engagement arm 1110 is configured to attach to the first spinal column segment by inserting the pin 1111 through the receiving opening and into the spinal column segment. The second engagement arm 1120 is configured to receive the second spinal column segment (e.g., sacral lamina) by moving the first and second tines 1122 and 1123 from an unfolded position to a folded position, as illustrated by the arrows in FIG. 8D. The coupling mechanism 1130 may comprise an expanding mechanism that may be adjusted to alter the spatial relationship between the two spinal column segments in order to achieve the desired degree of distraction. The dynamic portion 1140 is capable of contracting or expanding upon the application of biomechanical force within the body, thereby providing flexibility while maintaining proper spinal distraction.

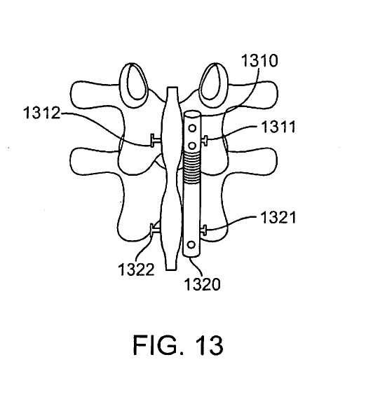

Alternatively, FIG. 12 shows the device comprising a first engagement arm 1210, a second engagement arm 1220, a coupling mechanism 1230, and a dynamic portion 1240. The first engagement arm comprises a fixation pin 1211 and a receiving opening (not shown) that is configured to receive the fixation pin 1211. The second engagement arm 1220 comprises projections such as at least two tines, for example, the first tine 1221 and the second tine 1222. During insertion, the first engagement arm 1210 is configured to attach to the first spinal column segment by inserting the pin 1211 through the receiving opening and into the spinal column segment. The second engagement arm 1220 is configured to receive the second spinal column segment (e.g., sacral lamina) between the tines 1221 and 1222. A further embodiment shown in FIG. 13 comprises a first engagement arm 1310 and a second engagement arm 1320. The first engagement arm 1310 may be attached to a spinal column segment such as the spinous process or the spinolaminar junction by inserting an attachment pin 1311 through a receiving opening (not shown) and into the spinal column segment. The attachment pin 1311 may then be locked in place by a locking cap 1312. The second engagement arm 1320 may be attached to another spinal column segment by inserting an attachment pin 1321 through a receiving opening (not shown) and into the spinal column segment. The attachment pin 1321 may then be locked in place by a locking cap 1322.

An alternative embodiment (not shown) similar to the one disclosed in FIG. 13 comprises a first engagement arm and a second engagement arm wherein the first and the second engagement arms terminate in U-shaped brackets. The U-shaped brackets are each shaped to receive the spinous processes of a spinal column segment. The U-shaped brackets may engage either unilaterally or bilaterally around the spinous processes.

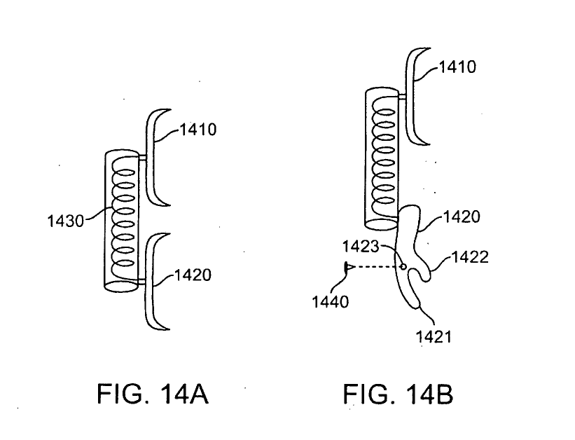

In another embodiment as shown in FIG. 14A, the device comprises a first engagement arm 1410, a second engagement arm 1420 and a coupling mechanism 1430. The first and the second engagement arms comprise clamp-like configurations that are configured to receive spinous processes of adjacent or non-adjacent spinal column segments. The coupling mechanism 1430 may be made of dynamic material or may comprise a spring or other load sharing mechanism. Alternatively, as shown in FIG. 14B, the second engagement arm 1420 may terminate in a sacrum receiving configuration, for example the second engagement arm 1420 comprises tines 1421 and 1422 configured to receive the sacrum. The second engagement arm 1420 in the embodiment shown in FIG. 14B may be secured for additional stability to the sacrum by using a screw 1440 received by a hole 1423 located on the second engagement arm 1420. Additionally, the engagement arm configuration exemplified in 1430 may be adapted to receive a lamina portion of the spinal column segment in addition to the sacrum.

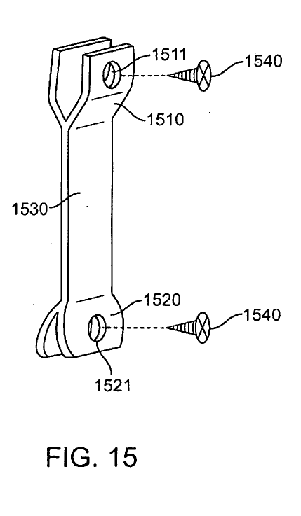

In another embodiment as shown in FIG. 15, the device comprises a first engagement arm 1510 and a second engagement arm 1520. The first and the second engagement arms 1510 and 1520 are configured to engage with the lamina portions of the spinal column segment. The first and the second engagement arms 1510 and 1520 may be secured to the spinal column segment by using screws 1540 received by receiving holes 1511 and 1521 located on the engagement arms. The receiving hole may be slotted as shown in 1511 or the receiving hole may be fitted as shown in 1521. The first engagement arm and the second engagement arms 1510 and 1520 are coupled together by a body 1530. The body 1530 may be rigid. During insertion, proper degree of distraction is first achieved by using methods and tools known to those skilled in the art, then the device embodied in FIG. 15 is inserted into the distraction site to maintain the distraction.

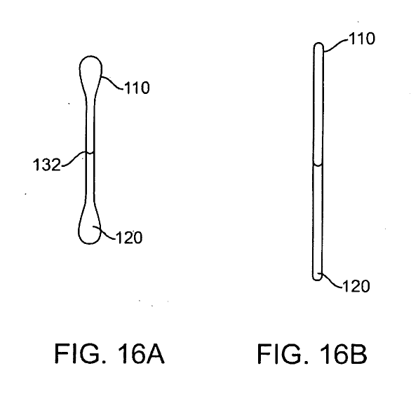

Additionally and optionally, as shown in FIG. 16A, the engagement arms of the device may comprise flared ends. Alternatively, as shown in FIG. 16B, the engagement arms of the device may comprise non-flared ends. Alternatively, one engagement arm may have a flared end while the other engagement arm may have a non-flared end (not shown). Arms 110 and 120 are exemplarily shown; however, any of the arms described here may have said flared or non-flared configurations. The flared and non-flared configurations are selected to produce a customized fit to a particular patient's vertebrae. The flared and non-flared configurations may also be altered and the width or angle customized to accommodate an individual patient's anatomy and pathology.

In another embodiment of the device, axial rotation of one or both ends of the device may be allowed to better accommodate the natural motion of the spine. This can be accomplished anywhere along the length of the device, either within an existing coupling mechanism or with a swiveling mechanism located at one or both ends of the device which allows the pronged ends of the device to rotate about the long axis of the device.

While the above is a complete description of the preferred embodiments of the invention, various alternatives, modifications, and equivalents may be used by those skilled in the art. Therefore, the above description should not be taken as limiting the scope of the invention which is defined by the appended claims.

What is claimed is:

1. A distracting device for distracting and stabilizing a spinal column segment, comprising:
a first engagement arm and a second engagement arm, wherein each arm is configured to engage with a lamina portion of the spinal column segment;
a coupling mechanism disposed between the first and the second engagement arms, wherein the coupling mechanism is configured to allow the device to transition from an unextended configuration to an extended configuration, wherein the device's transition from the unextended to the extended configuration causes the arms to distract the spinal column segment.

2. The device of claim 1, wherein the first engagement arm terminates in a lamina receiving configuration.

3. The device of claim 2, wherein the lamina receiving configuration comprises at least two tines configured to receive a first lamina portion of the spinal column segment.

4. The device of claim 1, wherein the second engagement arm terminates in a lamina receiving configuration.

5. The device of claim 4, wherein the lamina receiving configuration comprises at least two tines configured to receive a second lamina portion of the spinal column segment.

6. The device of claim 4, wherein the second engagement arm further comprises a secondary branch.

7. The device of claim 1, wherein the first engagement arm is configured to receive an attachment screw whereby the first engagement arm is attached to a vertebra.

8. The device of claim 1, wherein the first or second engagement arm is further secured to a lamina portion by a safety band.

9. The device of claim 1, wherein the first or the second engagement arm is further secured to a lamina portion by a screw.

10. The device of claim 1, wherein the coupling mechanism comprises a joint.

11. The device of claim 10, wherein the joint is a pivot joint, a revolute joint, a pin joint, or a hinge joint.

12. The device of claim 1, wherein the coupling mechanism comprises a telescoping mechanism, a slidable mechanism, a spring, or a rotation mechanism.

13. The device of claim 1 further comprises a locking mechanism configured to maintain the extended configuration of the device.

14. The device of claim 13, wherein the locking mechanism comprises a self-locking joint.

15. The device of claim 13, wherein the locking mechanism comprises a fastening screw and an opening configured to receive the fastening screw.

16. The device of claim 13, wherein the locking mechanism comprises a sleeve configured to be placed over the coupling mechanism.

17. The device of claim 1, wherein at least one portion of the device is dynamic.

18. The device of claim 1, wherein the distracting device is a part of a system comprising two distracting devices and a connecting mechanism, wherein the connecting mechanism is configured to connect the two distracting devices.

19. The device of claim 18, wherein the connecting mechanism comprises a U-hook, a process pin, or a safety band.

20. A device for distracting and stabilizing a spinal column segment, comprising:
a first engagement arm and a second engagement arm, the arms configured to engage with two or more lamina or sacral portions of the spinal column segment.

21. The device of claim 20, wherein the first engagement arm terminates in a lamina receiving configuration.

22. The device of claim 21, wherein the lamina receiving configuration comprises at least two tines configured to receive a first lamina portion of the spinal column segment.

23. The device of claim 20, wherein the second engagement arm terminates in a lamina receiving configuration.

24. The device of claim 23, wherein the lamina receiving configuration comprises at least two tines configured to receive a second lamina portion of the spinal column segment.

25. The device of claim 23, wherein the second engagement arm further comprises a secondary branch.

26. The device of claim 20, wherein the first engagement arm is configured to receive an attachment screw whereby the first engagement arm is attached to a vertebra.

27. The device of claim 20, wherein the first or second engagement arm is further secured to a lamina portion by a safety band.

28. The device of claim 20, wherein the first or the second engagement arms is further secured to a lamina portion by a screw.

29. The device of claim 20 further comprising a coupling mechanism disposed between the first and the second engagement arms.

30. The device of claim 29, wherein the coupling mechanism comprises a joint.

31. The device of claim 30, wherein the joint is a pivot joint, a revolute joint, a pin joint, or a hinge joint.

32. The device of claim 29, wherein the coupling mechanism comprises a telescoping mechanism, a slidable mechanism, a spring or a rotation mechanism.

33. The device of claim 20 further comprises a locking mechanism configured to maintain the extended configuration of the device.

34. The device of claim 33, wherein the locking mechanism comprises a self-locking joint.

35. The device of claim 33, wherein the locking mechanism comprises a fastening screw and an opening configured to receive the fastening screw.

36. The device of claim 33, wherein the locking mechanism comprises a sleeve configured to be placed over the coupling mechanism.

37. The device of claim 20, wherein a least one portion of the device is dynamic.

38. The device of claim 20 further comprises a connecting mechanism configured to connect said device to a second device.

39. The device of claim 38, wherein the connecting mechanism comprises a U-hook, a process pin, or a safety band.

40. A method for distracting a spinal column segment comprising the steps of:
positioning a device between a first spinal portion and a second spinal portion, wherein the device comprises:
a first engagement arm and a second engagement arm, the arms configured to engage with lamina or sacral portions of the spinal column segment;
a coupling mechanism disposed between the first and the second engagement arms, the coupling mechanism configured to allow the device to transition from an unextended configuration to an extended configuration,
wherein the device's transition from the unextended to the extended configuration causes the arms to distract the spinal column segment;
operating the coupling mechanism to extend the first and second engagement arms such that the engagement arms engage with the lamina portions of the first and second spinal portions to distract the spinal column segment.

41. The method of claim 40, further comprising separating the ligamentum flavum.

42. The method of claim 40, further comprising preparing the lamina portions for engagement.

43. The method of claim 40, further comprising sizing the desirable distraction.

44. The method of claim 40, further comprising securing the first and second engagement arms to lamina portions of the first and second spinal portions.

45. The method of claim 40, further comprising using a holder implement to secure the first and second engagement arms to lamina portions of the first and second spinal portions.

46. The method of claim 40, wherein the device further comprises a locking mechanism.

47. The method of claim 46, further comprising operating the locking mechanism to maintain the distraction

48. The method of claim 45, wherein securing the first and second engagement arms comprises the fitting lamina portions between the tines of the engagement arms.

49. The method of claim 45, wherein securing the first and second engagement arms comprises attaching a safety band to at least some portion of the first or second engagement arms.

50. The method of claim 45, wherein securing the first and second engagement arms comprises inserting a screw into one spinal column segment and a hole on the first or second engagement arms.

51. The method of claim 46, wherein operating the locking mechanism comprises engaging a self-locking joint.

52. The method of claim 46, wherein operating the locking mechanism comprises inserting a fastener screw into a receiving hole.

53. The method of claim 47, wherein operating the locking mechanism comprises placing a sleeve over the coupling mechanism.

54. The method of claim 40, further comprising using the device as a fusion adjunct.
